(19) United States
(12) Patent Application Publication
(10) Pub. No.: US 2017/0079931 A1
(43) Pub. Date: Mar. 23, 2017

(54) BODY TAPE HAVING AN ADHESIVE WITH COPPER PARTICLES

(71) Applicant: Intertape Polymer Corp., Sarasota, FL (US)

(72) Inventors: James Apap Bologna, Sarasota, FL (US); Ronald J. Horwitz, Palm Harbor, FL (US); John Paul Krug, Marysville, MI (US)

(21) Appl. No.: 15/267,238

(22) Filed: Sep. 16, 2016

Related U.S. Application Data
(60) Provisional application No. 62/220,303, filed on Sep. 18, 2015.

(51) Int. Cl. A61K 9/70; A61K 33/34

(52) U.S. Cl. CPC A61K 9/7061; A61K 33/34

(57) ABSTRACT

Adhesive tapes are disclosed that have a flexible backing conformable to a body part and have a top surface and a bottom surface and a layer of an adhesive composition disposed on the bottom surface of the flexible backing. The adhesive composition is formed from a pressure sensitive adhesive, and a copper metal powder and/or copper metal flakes having an apparent density in the range of 1 g/ml to 4 g/ml suspended throughout the adhesive composition, and may include a thickening agent.

### RELATED APPLICATIONS

This application claims the benefit of U.S. Provisional Application 62/220,303, filed Sep. 18, 2015, the entirety of which is incorporated herein by reference.

### FIELD OF THE INVENTION

The present invention relates to an adhesive tape having a pressure sensitive adhesive comprising copper particles, more particularly, to a body adhesive tape having a pressure sensitive adhesive comprising copper particles.

### BACKGROUND

Copper has some therapeutic effects, apparently including an aid in recovery of fatigued muscles and joints since micro strands of copper have been woven into tight fitting stretch fabrics for athlete garments. Copper Wear® high performance sportswear is an example of copper fiber embedded nylon fabric advertised as providing ease of movement while helping to relieve muscle and joint fatigue, soreness and stiffness. Since athletes are finding benefits from copper in their outer ware, they could also benefit from copper in a Kinesio® adhesive tape or other tape for application to a body part. There is a need to find a means to incorporate copper metal into a tape structure.

### SUMMARY

The adhesive tapes disclosed herein have copper metal incorporated into the tapes' structure, in particular in the adhesive layer of the tape. In all aspects, the adhesive tapes have a flexible backing conformable to a body part, the flexible backing having a top surface and a bottom surface, and have a layer of an adhesive composition disposed on the bottom surface of the flexible backing. The adhesive composition includes a pressure sensitive adhesive, copper metal powder and/or copper metal flakes having an apparent density in the range of 1 g/ml to 4 g/ml suspended throughout the adhesive composition, and, optionally, a thickening agent. The pressure sensitive adhesive is preferably a silicone adhesive or an acrylic adhesive.

When the pressure sensitive adhesive is the acrylic adhesive, the acrylic adhesive is either a solvent-less acrylic adhesive or a solvent-borne acrylic adhesive. When the pressure sensitive adhesive is a silicone adhesive, it is a solvent-borne adhesive.

With either the acrylic adhesive or the silicone adhesive, the copper metal powder and/or the copper metal flake has an apparent density of 1 g/ml and can have no thickening agent present or has thickening agent present. When the thickening agent is present, there is a thickening agent to copper metal ratio by weight of about 1:1 to 1:20, more preferably about 1:1 to about 1:8, and even more preferably about 1:2 to about 1:6. In the solvent-less adhesive compositions, a tackifier having a viscosity greater than 4000 cps/mPas may be present to function as the thickening agent, and the composition may include a wetting agent in a ratio of weight of about 1:2 to about 2:1 to the thickening agent, and a defoamer. In the solvent-borne adhesive compositions, the thickening agent and the copper metal powder and/or copper metal flake are present in a ratio of weights of about 1:1 to about 1:20, and the balance is typically the solvent-borne acrylic adhesive.

In all aspects of the adhesive tape, the flexible backing may be a film or a stretch base material, such as the backing used in Kinesio® adhesive tapes. The adhesive tapes may include a release structure applied to the top surface of the flexible backing and may include a barrier layer as an innermost layer in contact with the top surface of the flexible backing and a release layer as the outermost layer.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a perspective view of a roll of tape for one embodiment of the invention.

FIG. 2 is an enlarged cross-sectional view of the tape of FIG. 1, illustrating the layers thereof.

### DETAILED DESCRIPTION

The following detailed description will illustrate the general principles of the invention, examples of which are additionally illustrated in the accompanying drawings. In the drawings, like reference numbers indicate identical or functionally similar elements.

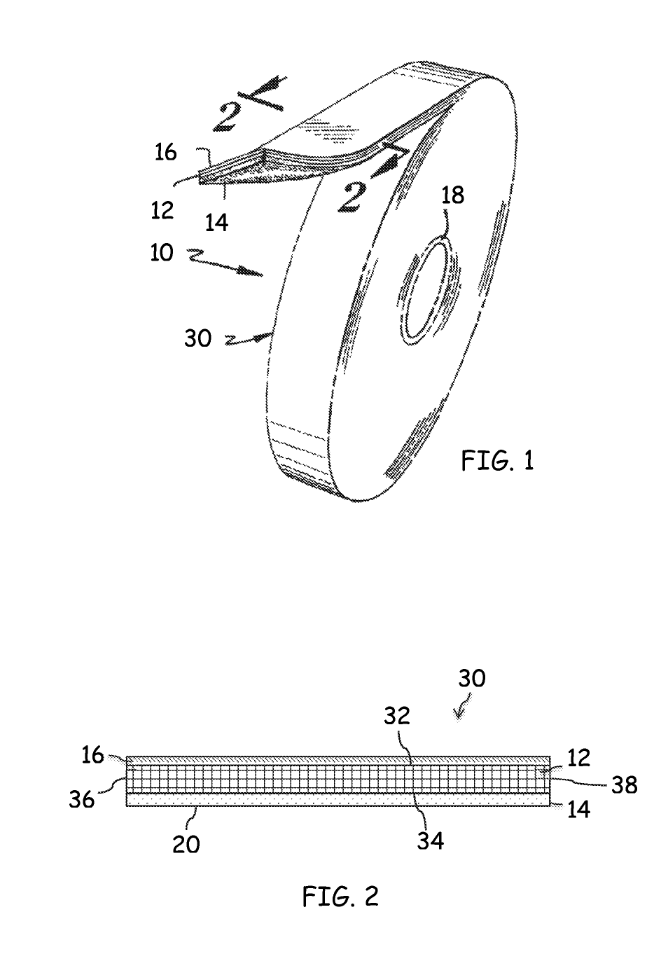

FIG. 1 illustrates a roll 10 of adhesive tape 30 wound onto a core 18 that includes an adhesive layer 14 that includes copper particles dispersed therein. The tape 30 as seen in the cross-section of FIG. 2 includes a plurality of layers applied to a backing 12 (also referred to as a substrate). The backing 12 has a top surface 32, a bottom surface 34, a first side 36, and a second side 38. Applied to the bottom surface 34 of the backing 12 is an adhesive layer 14, and applied to the top surface of the backing 12 is a release structure 16. The release structure 16 itself is optional and may include an optional barrier layer (not shown), which is present as a separate layer between the backing 12 and an outermost release layer 16. The release structure 16 is applied to the backing 12 on the surface opposite the adhesive layer 14 (the top surface with respect to the orientation of the figures with respect to the page).

Backing layer 12 may include natural and/or synthetic materials that are conformable to a body part of a person or animal. The backing layer 12 may be in the form of a foam, a polyolefin film, a polyurethane film, a cloth and/or stretch base material, woven, knit, or nonwoven materials, so long as the material is conformable to a body part. Examples of a cloth and/or stretch base material is described in U.S. Pat. No. 5,861,348 and U.S. Pat. No. 7,902,420. In one embodiment, the backing layer 12 may be a medical grade material that is conformable to a body part that is suitable for a backing layer and suitable to receive the adhesive on a major surface thereof, such as the bottom surface 34 in FIG. 2. In one embodiment, the backing layer 12 is a medical grade polyurethane film. Suitable medical grade polyurethane films are available from Mylan, Inc., under the trademark MEDIFILM®, as one example.

The optional barrier layer (not shown), if present, is disposed on backing 12 on the major surface opposite the adhesive layer 14, such as top surface 32 labeled in FIG. 2. One function of a barrier layer is to prevent the agents or chemicals contained in the release layer 26 from migrating into the backing 12. Likewise, in other embodiments, the barrier layer may be present on the bottom layer of the backing 12, e.g., between the adhesive layer 14 and the backing. Here, the copper particles included in the adhesive layer 14 are meant to stay in the adhesive layer, preferably more proximate the outermost surface 20 of the adhesive layer 14 than the backing 12, for contact with a body part, in particular, skin covering a body part. In another embodiment, if the backing 12 itself is not susceptible to absorbing the copper particles and/or other agents or chemicals in the adhesive composition defining the adhesive layer 14 or in the release layer 16, the barrier layers are unnecessary.

The release layer, if the release structure 16 is present, may be a silicone release coating, which allows the tape to be self-winding into rolls. The release layer is not limited to a silicone release coating. In other embodiments, any other suitable release system known to one of skill in the adhesive and/or tape art may be used. The thickness of the release structure 16 may be minimized relative to the thickness of the backing 12 and the adhesive layer 14 by applying the release structure 16 by any of the conventional printing, coating or air-brushing techniques known in the art. Thickness is one mechanism for maintaining the aesthetics and the performance of the tape 30 and minimizing the direct material cost thereof.

As shown in FIGS. 1-2, a pressure sensitive adhesive layer 14 is applied to the bottom surface 34 of backing 12. The adhesive layer 14 may be applied to backing 12 as an aqueous emulsion, a solvent-cast adhesive composition, or a solventless adhesive composition using any manner of roll, knife, rod, or blade coating, or curtain coating, or application by a lamination technique, or any other suitable method. In one embodiment, the adhesive coating layer thickness may range from about 0.1 mil to about 2.5 mil.

The pressure sensitive adhesive layer 14 is defined by an adhesive composition that includes an acrylic adhesive or a silicone adhesive, and copper metal powder or flake having an apparent density in the range of about 0.8 g/ml to about 8 g/ml suspended therein, more preferably about 1 g/ml to about 4 g/ml of copper metal powder or flake suspended therein. The pressure sensitive adhesive (PSA) when selected to include an acrylic adhesive may be a solvent-borne adhesive composition or a solvent-less adhesive composition.

In one aspect, the acrylic adhesive is a solvent-less acrylic adhesive. In one embodiment, the adhesive composition includes SYNTHEBOND® 7216 acrylic PSA emulsion available from Momentive Performance Materials Inc., which is a medical and transdermal grade acrylic PSA emulsion. Other suitable solvent-less acrylic adhesives include, but are not limited to, RODERM™ MID-5600 or RODERM™ MID-6000 acrylic PSAs, or a blend of both. These are butylacrylate/butyl-methacrylate-copolymer-dispersions and acrylic-acid copolymer-dispersions manufactured for skin-contact applications. Another suitable solvent-less acrylic adhesive is AROSET® 2022-W-50 thermosetting acrylic adhesive from Ashland Chemical of Columbus, Ohio.

When the adhesive composition includes the solvent-less acrylic adhesive, the adhesive composition may also have a tackifier. The tackifier may have a viscosity greater than 4000 cps/mPas and also function as the thickening agent. This adhesive composition may also include a wetting agent in a ratio of about 1:2 to about 2:1 relative to the thickening agent and/or tackifier functioning as a thickening agent. This adhesive composition may also include a defoamer.

In another aspect, the acrylic adhesive is a solvent-borne acrylic adhesive. In one embodiment, the adhesive composition includes DURO-TAK® 737NA acrylic PSA from Henkel Corporation. Many of the DURO-TAK® adhesives are developed for transdermal drug delivery and are therefore appropriate for skin contact of a body part. These include DURO-TAK® 129A, DURO-TAK® 1154 acrylic PSAs. Other suitable solvent-borne acrylic adhesives include AROSET® 1196 medical-grade acrylic polymer adhesive from Ashland. Chemical of Columbus, Ohio.

When the adhesive composition includes the silicone pressure sensitive adhesive, it is a solvent-borne adhesive. Example adhesives include, but are not limited to, Dow Corning® MG-2401 silicone pressure sensitive adhesive with hexamethyl disiloxane as the solvent, Dow Corning® MG-2402 silicone pressure sensitive adhesive with ethyl acetate as the solvent, Dow Corning® MG-2502 silicone pressure sensitive adhesive with ethyl acetate as the solvent, and Dow Corning BIO-PSA 7-4202, 7-4402, and 7-4502 silicone adhesive in either heptane or ethyl acetate.

Thickening agents may be added to the solvent-borne acrylic adhesive or the solvent-borne silicone adhesive to disperse the copper metal powder or flake throughout the adhesive composition. The addition of a thickening agent is generally helpful when the apparent density of the copper metal is greater than 1 g/ml. The thickening agent and copper metal powder have a ratio of weight of about 1:1 to about 1:20, more preferable about 1:1 to about 1:8. In another embodiment, this ratio may be about 1:2 to about 1:6. In one embodiment, the thickening agent includes CAB-O-SIL® M-5, an untreated fumed silica from the Cabot Corporation, SOLTHIX® 250, a methoxyl propyl acetate-containing thickening agent from Lubrizol, Byk-431 or BYK Garamite 1958 thickening agents from BYK USA Inc., and combinations thereof.

Copper metal powder is readily available from various manufacturers. One example is CU 301E copper from OZO Metals Electrolytic Copper Powder at 325 mesh, having an apparent density of 1.5 g·cm⁻³. Another example is SCM Metal Products, Inc. high green strength copper powders available as 180RLX-HGS with an apparent density of 2.15 g/cm³, 200RLX-HGS with an apparent density of 1.7 g/cm³, 285RLX-HGS with an apparent density of 1.38 g/cm³, and 400RLX-SGS with an apparent density of 1.00 g/cm³. Another example is copper metal powder from MiniScience, Inc. of Clifton, N.J. having an apparent density of 2.97 g/ml, which was used in the Example below.

Copper metal flakes may be present in the adhesive composition, with or without the copper metal powder, in sizes from 100 mesh to 325 mesh available from any manufacturer or supplier.

### EXAMPLE 1

### Solvent-Less Adhesive

Copper metal powder was obtained from MiniScience, Inc. of Clifton, N.J. The copper metal powder was described as having an apparent density of 2.97 g/ml and as comprising microparticles. The copper metal powder was dispersed in a solvent-less acrylic adhesive composition according to the formulation below.

TABLE 1

| | | Wet weight | |
|---|---|---|---|
| Ingredient | Commercial Name | (g) | % wt/wt |
| Acrylic adhesive | MOMENTIVE ® | 100.0 g | 90.1 |
| | SYNTHEBOND ® 7216 | | |
| Thickening Agent | BASF TB-15 | 3.0 g | 2.7 |
| Foam Control Agent | DREWPLUS ™ Y-281 | 1.0 g | 0.9 |
| Wetting | SURFYNOL ® PSA336 | 1.0 g | 0.9 |
| Agent/Surfactant | | | |
| Source of copper | CU 301E Copper Powder | 6.0 g | 5.4 |

The SURFYNOL® PSA336 was obtained from Air Products and Chemicals, Inc. The adhesive composition was prepared by mixing the components listed in Table 1 at low shear for 3 minutes (order of addition is not important).

MEDIFILM® 426 and 437, clear 1.0 Mil polyurethane films, were obtained from Mylan Inc. to be the backing of the adhesive tape. These are medical grade films.

After mixing, the adhesive composition was coated onto a 4 mil siliconized liner at a thickness of approximately 3 mil. The adhesive composition, on the siliconized liner, was dried in a forced-air oven for 3 minutes at 80° C. followed by 3 minutes at 108° C. Then, some of the adhesive composition was transferred from the siliconized liner sheet onto the MEDIFILM® 426 polyurethane film and onto the MEDIFILM® 437 polyurethane film using a Cheminstruments Laboratory Laminator. Each of these polyurethane films was laminated with the adhesive composition on the smooth side thereof and the lightly textured side facing up (no adhesive composition on this opposing side).

### EXAMPLE 2

### Solvent-Less Adhesive

Copper metal powder was obtained from MiniScience, Inc. of Clifton, N.J. The copper metal powder was described as having an apparent density of 2.97 g/ml and as comprising microparticles. The copper metal powder was dispersed in a solvent-less acrylic adhesive composition according to the formulation below.

TABLE 2

| Ingredient | Commercial Name | % wt/wt |
|---|---|---|
| Acrylic adhesive | BASF ACRONAL ® A220 | 97 |
| Thickening Agent | BASF TB-15 | 0.3 |
| Foam Control Agent | BYK-016 | 0.3 |
| Wetting | SURFYNOL ® PSA336 | 0.5 |
| Agent/Surfactant | | |
| Source of copper | CU 301E Copper Powder | 1.9 |

The adhesive composition was prepared by mixing the components listed in Table 1 at low shear for 3 minutes (order of addition is not important), and was thereafter coated onto a 4 mil siliconized liner at a thickness of approximately 3 mil. The adhesive composition was dried according to the procedure in Example 1. Then, some of the adhesive composition was transferred from the siliconized liner sheet onto the smooth side of MEDIFILM® 426 polyurethane film and MEDIFILM® 437 polyurethane film using a Cheminstruments Laboratory Laminator.

### EXAMPLE 3

### Solvent-Borne Adhesive

A suitable solvent-borne adhesive composition was formulated from DURO-TAK® AH-115 from Henkel Corporation and thickening agents. The thickening agents enable the formation of a stable dispersion of the copper metal powder in a solvent based adhesive, such that the copper does not settle out of the adhesive composition. An example adhesive composition for a solvent-based version is given in Table 3 below.

TABLE 3

| Ingredient | Commercial Name | Wet weight (g) | % wt/wt |
|---|---|---|---|
| solvent-borne | DURO-TAK ® 737NA | 100 | 82.6 |
| acrylic adhesive | | | |
| Thickening agent | BYK GARAMITE | 1 | 0.9 |
| | 1958 | | |
| Source of copper | CU 301E Copper | 20 g | 16.5 |
| | Powder | | |

This adhesive composition includes a thickening agent as noted in Table 3. The adhesive composition was prepared by mixing the components listed in Table 3 at low shear for 3 minutes (order of addition is not important).

MEDIFILM® 426 and 437, clear 1.0 Mil polyurethane films, were obtained from Mylan Inc. to be the backing of the adhesive tape. These are medical grade films.

After mixing, the adhesive composition was coated onto a 4 mil siliconized liner at a thickness of approximately 3 mil. The adhesive composition, on the siliconized liner, was dried in a forced-air oven for 3 minutes at 80° C. followed by 3 minutes at 108° C. Then, some of the adhesive composition was transferred from the siliconized liner sheet onto the MEDIFILM® 426 polyurethane film and onto the MEDIFILM® 437 polyurethane film using a Cheminstruments Laboratory Laminator. Each of these polyurethane films was laminated with the adhesive composition on the smooth side thereof and the lightly textured side facing up (no adhesive composition on this opposing side).

Applicants theorize that the solvent-borne adhesive composition may have some advantages, in particular that the composition may improve shelf life and reduce the amount of discoloration of the copper that results from oxidation of the copper.

Tape samples having a solvent-borne acrylic adhesive with a higher density copper (5 g/ml) made according to the procedure of Example 3 were prepared to have copper as about 50 parts per hundred of the adhesive. The tape samples were tested for antimicrobial activity and efficacy utilizing test method JIS Z 2801 designed to assess the performance of antimicrobial finishes on hard, non-porous surfaces. Staphylococcus aureus (a gram-positive anaerobe), Escherichia coli (a gram-negative anaerobe), and Candida albicans were tested on separate tape samples. Each of these microorganisms were separately prepared, standardized by a dilution in a nutritive broth, and applied to the adhesive surface of test size samples (50 mm×50 mm) of the tape under the standardized conditions of JIS Z 2801. Three samples for each microorganism were evaluated after 24 hours as compared to a control (tape sample with same adhesive formulation, but no copper) to determine the microbial concentration reduction relative to the control. The tape was successful at reducing the presence of the S. Aureus and E. Coli to greater than 99.9% and the presence of C. albicans by about 98.6%, a log₁₀ of 1.88 as compared to the control. Thus, the advantage of the tape is the adhesive formulation and its ability to greatly reduce antimicrobial growth.

Having described the invention in detail and by reference to preferred embodiments, it will be apparent that modifications and variations thereof are possible without departing from the scope of this invention. The tape is preferably a body adhesive tape or a bandage, but is not limited thereto.

What is claimed:

1. An adhesive tape comprising:
a flexible backing conformable to a body part and having a top surface and a bottom surface; and
a layer of an adhesive composition disposed on the bottom surface of the flexible backing, the adhesive composition comprising:
a pressure sensitive adhesive;
copper metal powder and/or copper metal flakes having an apparent density in the range of 1 g/ml to 4 g/ml suspended throughout the adhesive composition; and
optionally, a thickening agent.

2. The adhesive tape of claim 1, wherein the pressure sensitive adhesive is a silicone adhesive or an acrylic adhesive.

3. The adhesive tape of claim 2, wherein the pressure sensitive adhesive is the acrylic adhesive, and the acrylic adhesive is a solvent-less acrylic adhesive.

4. The adhesive tape of claim 1, wherein the copper metal powder and/or the copper metal flake has an apparent density of 1 g/ml and no thickening agent is present.

5. The adhesive tape of claim 1, wherein the copper metal powder and/or copper metal flake has an apparent density greater than 1 g/ml and the thickening agent is present in a thickening agent to copper metal ratio of weight of about 1:1 to 1:20.

6. The adhesive tape of claim 5, wherein the thickening agent and copper metal are present in a ratio of weight of about 1:1 to about 1:8.

7. The adhesive tape of claim 5, wherein the thickening agent and the copper metal are present in a ratio of weight of about 1:2 to about 1:6.

8. The adhesive tape of claim 5, wherein the adhesive composition further comprises a tackifier having a viscosity greater than 4000 cps/mPas functioning as the thickening agent.

9. The adhesive tape of claim 5, wherein the adhesive composition further comprises a wetting agent in a ratio of weight of about 1:2 to about 2:1 with the thickening agent.

10. The adhesive tape of claim 8, wherein the adhesive composition further comprises a defoamer.

11. The adhesive tape of claim 1, wherein the acrylic adhesive is a solvent-borne acrylic adhesive.

12. The adhesive tape of claim 8, wherein the thickening agent and the copper metal powder and/or copper metal flake are present in a ratio of weight of about 1:1 to about 1:20, and the balance is the solvent-borne acrylic adhesive.

13. The adhesive tape of claim 1, wherein the flexible backing is a film.

14. The adhesive tape of claim 1, wherein the flexible backing is a stretch base material.

15. The adhesive tape of claim 1, further comprising a release structure applied to the top surface of the flexible backing.

16. The adhesive tape of claim 1, wherein the release structure comprises a barrier layer as an innermost layer in contact with the top surface of the flexible backing and a release layer as the outermost layer.
